(19) United States
(12) Patent Application Publication
(10) Pub. No.: US 2025/0098957 A1
(43) Pub. Date: Mar. 27, 2025

(54) INTRAOCULAR PRESSURE MEASUREMENT SYSTEM AND METHOD

(71) Applicant: NogalMedicine GmbH, Visp (CH)

(72) Inventors: Piotr Zbigniew Nogal, Glis (CH); Anna Grazyna Nogal, Glis (CH)

(21) Appl. No.: 18/897,818

(22) Filed: Sep. 26, 2024

Related U.S. Application Data
(60) Provisional application No. 63/540,521, filed on Sep. 26, 2023.

(51) Int. Cl. A61B 3/16; A61B 3/10; A61B 3/107; A61B 3/14

(52) U.S. Cl. CPC A61B 3/16; A61B 3/1005; A61B 3/107; A61B 3/14

(57) ABSTRACT

A system for measuring intraocular pressure (IOP). The system includes a tonometer head configured to measure IOP in a posterior chamber and a vitreous cavity of an eye. The system includes an optical gauge configured to measure corneal data of the eye. The system includes a compression arm-spread angle gauge configured to measure a dilation angle of compression arms. The system includes a processor configured to determine geometric parameters of the eye based on the measured corneal data and dilation angle, calculate physical parameters of a sclera of the eye based on the determined geometric parameters, and adjust the measured IOP based on the calculated physical parameters of the sclera.

### RELATED APPLICATION DATA

This application claims priority to U.S. Provisional Patent Application Ser. No. 63/540,521 filed on Sep. 26, 2023, the entire disclosure of which is incorporated herein by reference.

### BACKGROUND

Intraocular pressure (IOP) measurement is a critical component of ophthalmic examinations, particularly for the diagnosis and management of glaucoma. Traditionally, tonometry techniques have focused on measuring IOP in the anterior chamber of the eye, typically through the cornea. These methods include applanation tonometry, non-contact tonometry, and rebound tonometry, among others.

While anterior chamber IOP measurements have been the standard of care for many years, there is growing recognition that pressure differentials can exist between the anterior and posterior chambers of the eye. This is particularly relevant in cases of pupillary block, where the flow of aqueous humor from the posterior to the anterior chamber may be impeded, potentially leading to elevated pressure in the posterior segment without a corresponding increase in anterior chamber pressure.

The posterior segment of the eye, including the vitreous cavity and structures such as the retina and optic nerve head, is particularly susceptible to damage from elevated IOP. However, directly measuring pressure in these areas has historically been challenging due to their anatomical location and the desire to avoid invasive procedures.

Additionally, the relationship between IOP and the risk of glaucomatous damage is complex and can vary between individuals. Factors such as corneal thickness, scleral rigidity, and overall eye size may influence how a given IOP level affects the optic nerve and other ocular structures. This variability highlights the need for more comprehensive and personalized approaches to IOP assessment.

Current tonometry methods also face limitations in certain clinical scenarios, such as in patients with corneal abnormalities or those who have undergone corneal surgeries. These situations can affect the accuracy of traditional IOP measurements and may necessitate alternative approaches.

As scientific understanding of ocular physiology and the pathophysiology of glaucoma continues to evolve, there is an increasing need for more sophisticated and comprehensive IOP measurement techniques. Ideally, such methods would provide information not only about the pressure in different compartments of the eye but also about the individual anatomical and biomechanical factors that may influence the clinical significance of a given IOP level.

### SUMMARY OF INVENTION

According to an aspect of the present disclosure, a system for measuring intraocular pressure (IOP) is provided. The system includes a tonometer head configured to measure IOP in a posterior chamber and a vitreous cavity of an eye. The system includes an optical gauge configured to measure corneal data of the eye. The system includes a compression arm-spread angle gauge configured to measure a dilation angle of compression arms. The system includes a processor configured to determine geometric parameters of the eye based on the measured corneal data and dilation angle, calculate physical parameters of a sclera of the eye based on the determined geometric parameters, and adjust the measured IOP based on the calculated physical parameters of the sclera.

According to other aspects of the present disclosure, the system may include one or more of the following features. The optical gauge may comprise a keratometer configured to measure corneal curvature. The optical gauge may comprise a pachymeter configured to measure corneal thickness. The compression arm-spread angle gauge may be configured to measure the dilation angle when compression feet of the compression arms contact the eye at a predetermined distance from a corneal limbus. The predetermined distance may be between 3-5 mm posterior to the corneal limbus. The system may further comprise an axial drive configured to maintain the optical gauge at a constant distance from a cornea of the eye during measurement. The system may further comprise a camera configured to capture an image of an anterior surface of a lens of the eye to determine a degree of porosity of an anterior lens capsule.

According to another aspect of the present disclosure, a method of measuring intraocular pressure (IOP) is provided. The method includes measuring IOP in a posterior chamber and a vitreous cavity of an eye using a tonometer. The method includes measuring corneal data of the eye using an optical gauge. The method includes measuring a dilation angle of compression arms using a compression arm-spread angle gauge. The method includes determining geometric parameters of the eye based on the measured corneal data and dilation angle. The method includes calculating physical parameters of a sclera of the eye based on the determined geometric parameters. The method includes adjusting the measured IOP based on the calculated physical parameters of the sclera.

According to other aspects of the present disclosure, the method may include one or more of the following features. Measuring the corneal data may comprise measuring corneal curvature using a keratometer. Measuring the corneal data may further comprise measuring corneal thickness using a pachymeter. Measuring the dilation angle may comprise measuring the dilation angle when compression feet of the compression arms contact the eye at a predetermined distance from a corneal limbus. The method may further comprise maintaining an optical gauge at a constant distance from a cornea of the eye during measurement using an axial drive. The method may further comprise capturing an image of an anterior surface of a lens of the eye to determine a degree of porosity of an anterior lens capsule.

According to another aspect of the present disclosure, a non-transitory computer-readable medium storing instructions is provided. When executed by a processor, the instructions cause the processor to perform a method of measuring intraocular pressure (IOP). The method includes receiving IOP measurements from a posterior chamber and a vitreous cavity of an eye. The method includes receiving corneal data measurements of the eye. The method includes receiving a dilation angle measurement of compression arms. The method includes determining geometric parameters of the eye based on the received corneal data and dilation angle. The method includes calculating physical parameters of a sclera of the eye based on the determined geometric parameters. The method includes adjusting the received IOP measurements based on the calculated physical parameters of the sclera.

According to other aspects of the present disclosure, the non-transitory computer-readable medium may include one or more of the following features. The corneal data measurements may comprise corneal curvature and corneal thickness. The method may further comprise receiving an image of an anterior surface of a lens of the eye. The method may further comprise determining a degree of porosity of an anterior lens capsule based on the received image. Calculating the physical parameters of the sclera may comprise determining a thickness, axial stiffness, and bending stiffness of the sclera. Adjusting the received IOP measurements may comprise applying a correction factor based on the determined thickness, axial stiffness, and bending stiffness of the sclera.

### BRIEF DESCRIPTION OF THE DRAWING

FIG. 1 is a schematic illustration of normal blood flow between the brain (cavernosus sinus) and eye at normal intraocular pressure (15 mmHg) in the posterior chamber of the eye and vitreous cavity.

FIG. 2 illustrates Intraocular pressure differentiation in the ocular chambers in increased pupillary block, where outflow of the aqueous humor occurs passively due to the pressure gradient between the chambers of the eye, providing evidence of higher pressure in the posterior chamber than in the anterior chamber.

FIG. 3 is a schematic illustration of pathological blood flow between the brain and eye at pupillary block with secondary increased IOP in posterior chamber and vitreous cavity (40 mmHg) and normal pressure (15 mmHg) in the anterior chamber, where conventional IOP measurements are made.

FIG. 4a shows a situation of normal IOP=20 mmHg in the emmetropic eyeball (normal eye size); hydraulic vessels of normal length and diameter. Blood and CSF flow inside the eyeball and between eye and brain normal. Normal IOP is not dangerous for the eye and brain; IOP lower than in hydraulic vessels in the eye.

FIG. 4b shows a situation of normal IOP=20 mmHg in the myopic eyeball (enlarged eye) higher than a pressure of blood and CSF in stretched hydraulic vessels with increased length, reduced diameter and decreased hydrostatic pressure (Bernoulli paradox).

FIG. 5 illustrates an example of a known tonometer.

FIG. 6 illustrates and example of a tonometer in accordance with disclosed implementations.

FIG. 7 is a diagram illustrating measurement of the geometry of the eyeball in accordance with disclosed implementations.

FIG. 8 illustrates the various angular and linear quantities measured by the tonometer in accordance with disclosed implementations.

FIG. 9 illustrates a flowchart for a method of measuring and adjusting intraocular pressure (IOP)

### DETAILED DESCRIPTION

A very common type of glaucoma is knowns as “normal pressure glaucoma” or “normal tension glaucoma” (NTG). Moreover, most of the tonometers used today are used to measure IOP on the cornea, i.e., in the anterior chamber of the eye. However, e structures subject to pathology due to intraocular hypertension are located behind the lens and iris, i.e. in the space of the posterior chamber and the vitreous cavity. Pressure in the posterior chamber affects these structures of the fundus of the eye (optic nerve head, retinal nerve fibre layer (RNFL), choroid and intraocular blood vessels) connected with brain. There is scientific evidence of differential IOP in the two chambers of the eye.

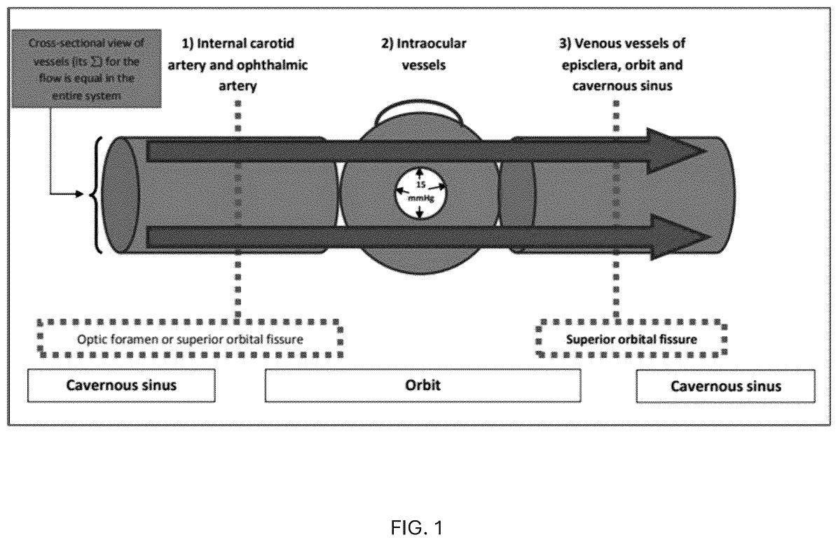

The common hydraulic vessels of the brain and eye transporting fluids into the eyeball that occur behind the lens and iris provide the opportunity for indirect and noninvasive measurement of intracranial pressure by measuring IOP in these spaces. These include the blood vessels of the retina, choroid, axoplasmic channels of the optic nerve fibers and the subarachnoid space of the brain connected to the corresponding space between the optic nerve meninges. FIG. 1 schematically illustrates normal blood flow between the brain (cavernosus sinus) and eye at normal intraocular pressure (15 mmHg) in the posterior chamber of the eye and vitreous cavity.

IOP is regulated by quantitative parameters (rate and volume) of the production and outflow of aqueous humor in the eye. A significant role is also played by qualitative parameters, such as density and viscosity, which are individually variable. The aqueous humor is produced in the posterior chamber of the eye and must outflow through the pupil into the anterior chamber. From the anterior chamber, it flows through the anterior chamber angle into the orbital veins and further into the cerebral venous system in the cavernous sinus.

The differentiation of IOP in the two chambers is compounded by the presence of physiological pupillary block, i.e. blocking of the pupil from behind by the lens growing till the end of life, which always to some extent inhibits the outflow of aqueous humor through the pupil. This is evidenced by backward deflection of the iris during dynamic gonioscopy, when the gonioscope compresses the cornea more strongly, temporarily increasing the pressure in the anterior chamber, which nevertheless does not allow the fluid to flow back into the posterior chamber. This situation provides evidence of the physiologic pupillary block, a “pupillary valve” that blocks the backflow of the aqueous humor to the posterior chamber similar to the venous valves that prevent the backflow of blood in them.

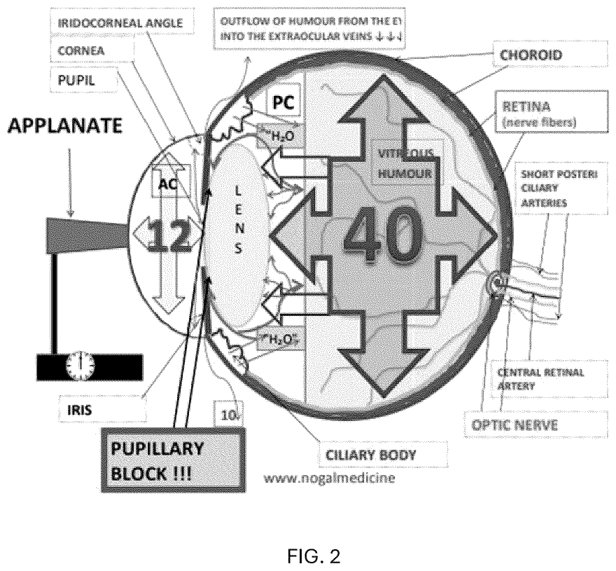

Increased pupillary block is a common pathophysiological situation that causes significantly ocular pressures in both eye chambers. It leads to an increase in pressure in the posterior chamber, where aqueous humor is produced, with a secondary decreased, stable or increased pressure in the anterior chamber, which is merely a pathway for the outflow of aqueous fluid from the eyeball into the orbital veins and brain. It's possible that anterior chamber IOP is higher, than posterior chamber IOP. Conventional IOP measurements made on the cornea, which is the anterior wall of the anterior chamber of the eye, are inadequate in this situation to measure the IOP prevailing behind the blocked pupil, that is, in the posterior chamber and vitreous cavity (which is the most important risk factor of a glaucoma, when increased). FIG. 2 illustrates Intraocular pressure differentiation in the ocular chambers in increased pupillary block, where outflow of the aqueous humor occurs passively due to the pressure gradient between the chambers of the eye, providing evidence of higher pressure in the posterior chamber than in the anterior chamber.

It has also been observed by the inventors that the anterior surface of the lens becomes smoother in patients as they age (due to the growth of the lens, the anterior capsule of the lens also tightens by the end of life and becomes stretched and smoother). In glaucoma patients, the anterior capsule that previously looked like an “orange peel” is almost completely smoothed out, causing it to adhere more firmly to the iris from behind and thus blocking the pupil to an even greater degree. The hypothesis of the inventors is that a decrease in anterior lens surface porosity is responsible for exacerbating pupillary block to a greater extent than axial lens growth till the end of life. Increased posterior IOP leads to impaired blood and aqueous humor flow between brain and eye, also CSF in the optic nerve, resulting in increased orbital and cerebral intracranial pressure (ICP) with possible central nervous system (CNS) complications due to neurovascular conflicts.

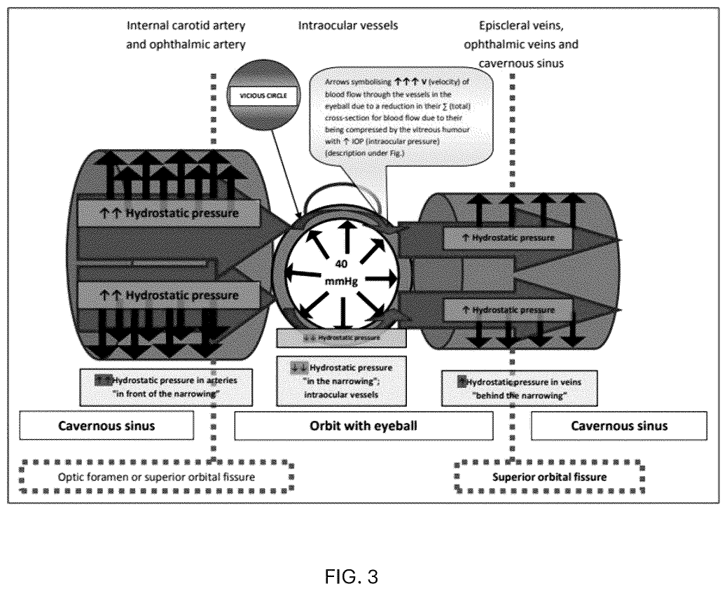

FIG. 3 illustrates pathological blood flow between the brain and eye at pupillary block with secondary increased IOP in posterior chamber and vitreous cavity (40 mmHg) and normal pressure (15 mmHg) in the anterior chamber, where conventional IOP measurements are made. According to Bernoulli's rule, there is an increase in hydrostatic pressure upstream and downstream of the obstruction to flow. There is a paradoxical decrease in hydrostatic pressure at the stenosis site, which leads to even easier hydraulic vessel closure at the stenosis site by increased IOP. FIG. 3 shows that the tonometer described in this application, which measures IOP in the posterior chamber and vitreous cavity, has the potential to measure ICP non-invasively.

The aqueous fluid secreted in the eye is largely Cerebrospinal Fluid (CSF), which flows from the brain into the eye through the optic nerve fibers and the subarachnoid space. CSF is a clear, colorless liquid that surrounds the brain and spinal cord, providing cushioning and protection. This represents, according to the inventor, the sought-after most important pathway for CSF outflow from the brain. The CSF that is part of the aqueous humor in the eye is to nourish the intraocular structures (nerve fibers, retinal cells, vitreous body, lens and cornea).

This is evidenced by the anatomy and physiology of the optic nerve structure and such disease syndromes as “PEX” syndrome (pseudoexfoliation syndrome; amyloid the same as in the brain appears in the eyeball causing glaucoma or epiretinal membrane) or Terson's syndrome (subarachnoid hemorrhage in the brain is accompanied by hemorrhage into the vitreous body without any other detectable changes in the eye). The route of CSF outflow from the brain to veins through the cerebral arachnoid granulations, assumed so far, is illogical as it assumes the outflow of CSF continuously produced already in human fetus through the structures, which often appear in the brain only after the child is 3 years old. This would mean that there is no possibility of CSF outflow from the brain until this age and hydrocephalus develops in every human being; however, this does not happen.

So far, it has been believed in ophthalmology that 20% of the aqueous humor is produced passively and 80% actively by unknown humoral mechanisms. The inventors posit that this is incorrect because the mechanism of aqueous humor production has not been established. Evidence that the opposite is probably true is that all hypotensive drugs applied topically to the eye in glaucoma lower IOP by up to 25%. The drug with the highest IOP-lowering effect (up to 75%) is generally administered mannitol or acetazolamide (inhibiting the production of CSF in the brain), which are used in neurology to lower cerebral pressure (ICP), e.g., in cerebral edema.

The hypothesis of the inventors, confirmed by the anatomy of a child's eye and brain, is the role of physiologically enhanced pupillary block in the development of the eyeball. In the “BIG Eye” (Block Induced Growth of Eye) paper, the authors of the invention described the physiological mechanism of increased pressure in the posterior chamber and vitreous body in the child's eye by increased pupillary block, which leads to eyeball swelling by the prevailing ocular hypertension and an increase in the size of the eyeball resembling an inflated rubber balloon.

The enlargement of the eyeball during the first year of a child's life allows the axial dimension of the eyeball to increase from 14 to 21 mm, which, according to the authors, is the hydraulic and most important mechanism responsible for the emmetropization of the eye from hyperopia to emmetropia. Emmetropization is the process by which the eye develops to achieve optimal vision, specifically focusing on reducing refractive errors like myopia (nearsightedness) and hyperopia (farsightedness). This process typically occurs from birth through childhood, as the eye's components adjust to ensure that light is properly focused on the retina. In newborns, the eyes are usually slightly hyperopic (farsighted), but as they grow, the axial length of the eye increases, and the refractive power of the cornea and lens adjusts to bring the eye closer to emmetropia, where distant objects are in sharp focus without the need for corrective lenses.

According to the authors, overdriving of this mechanism in the face of excessive accommodation in the normal-sighted eye is the reason for the onset and progression of myopia. The essence of myopia is enlargement of the eyeball dimensions beyond the norm, which due to the formation of the image in front of the retina, requires the use of light-diffusing spherical correction; minus. The inventors' hypothesis is that during excessive accommodation, there is:
constriction of the pupil in response to accommodation with increased pupillary block and secondary increase in IOP behind the lens and iris; in the posterior chamber and vitreous body,
functional deformation of the eyeball with its stretching in the sagittal axis by the ciliary muscle,
enlargement of all dimensions of the eyeball (axial dimension, circumference, wall surface, volume, corneal diameter),
a secondary to the above, thinning of the eyeball walls,
additional to the above, a decrease in the axial stiffness of the sclera, allowing it to stretch further and more easily,
a secondary to this, elongation of the hydraulic vessels for fluids (CSF and blood) entering the eyeball from the cerebral circulation,
a secondary decrease in the diameter of these hydraulic vessels in the fundus, which leads according to Bernoulli's paradox to an acceleration of flow with a decrease in hydrostatic pressure at the site of constriction,
the secondary to the above easier closure of these hydraulic vessels by even normal IOP, which leads to an increase in hydrostatic pressure greatest before the obstruction to flow and less behind it, which amounts to an increase in pressure in the orbit and brain.

According to the inventors, this is the only way to enlarge the area of the macula, which grows with the entire eyeball due to physiological hypertension inside it. So we have a situation in ophthalmology and neurology where, in different sized eyeballs, the pressure critical for the onset and development of eye and brain disorders will present different IOP values. This requires technology that allows personalization of IOP measurement not only for an individual patient, but often for each eye separately (e.g., in anisometropia, where one eye may differ significantly in size from the other).

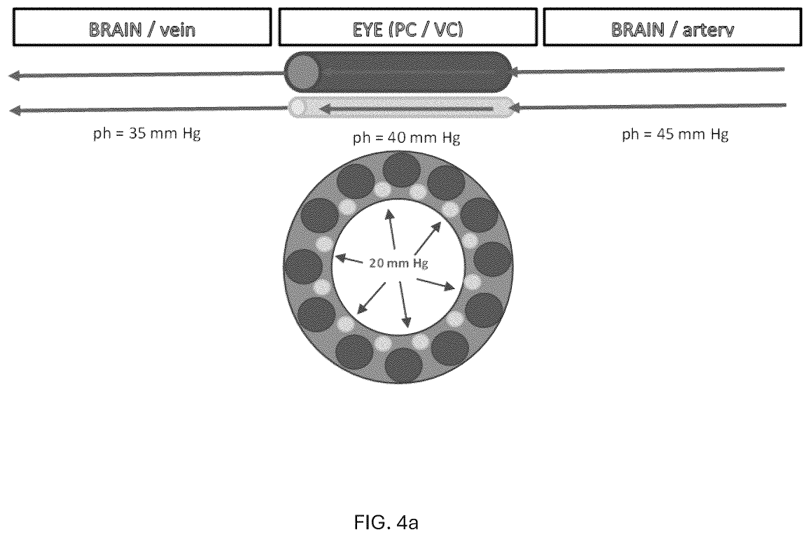

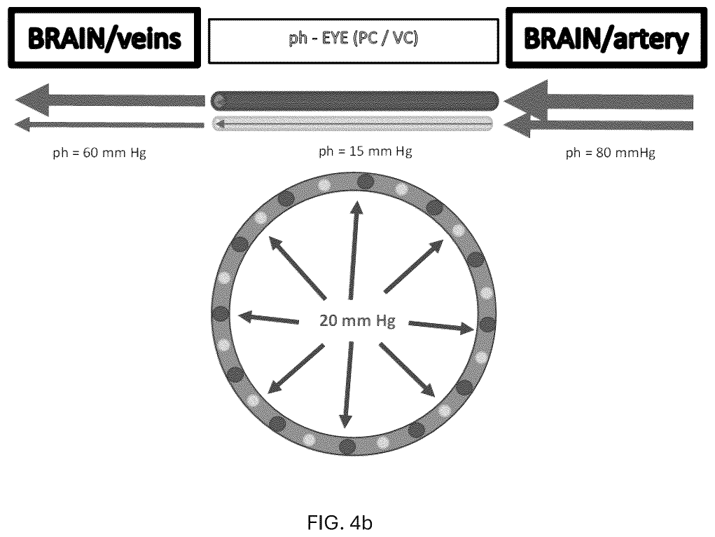

As shown in FIGS. 4a and 4b, blood and CSF flow between the eye and brain in different sized eyeballs at the same IOP and the different effects of the same pressure on the onset of disease disorders, where:
Red hydraulic vessels—blood vessels
Yellow hydraulic vessels—axoplasmic spaces of optic nerve fibers leading CSF
Blue arrows—flow of fluids between brain and eye (from the brain through the eye back to the brain)

FIG. 4a shows a situation of normal IOP=20 mmHg in the emmetropic eyeball (normal eye size); hydraulic vessels of normal length and diameter. Blood and CSF flow inside the eyeball and between eye and brain normal. Normal IOP is not dangerous for the eye and brain; IOP lower than in hydraulic vessels in the eye. FIG. 4b shows a situation of normal IOP=20 mmHg in the myopic eyeball (enlarged eye) higher than a pressure of blood and CSF in stretched hydraulic vessels with increased length, reduced diameter and decreased hydrostatic pressure (Bernoulli paradox). Usually normal pressure is dangerous for the eye and brain. Blood and CSF flow between eye and brain disturbed. The thicker blue arrows of the flow symbolize the increase in hydrostatic pressure with decrease in flow velocity according to Bernoulli's rule. Normal IOP is not dangerous for the eye and brain; IOP lower than in hydraulic vessels in the eye.

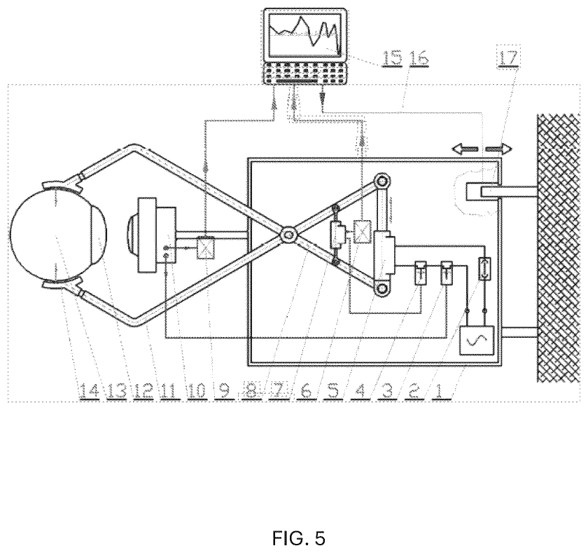

FIG. 5 illustrates the tonometer disclosed in WO2015/114446. Head 1 is axially movable with respect to the body by distance actuators 17, which allow head 1 to be moved axially closer to or further from the eye of the patient. Computer 15 sends control signals to actuators 17 to affect the movement as appropriate for each patient. Head 1 includes impression actuator 5, mode switch 2, automatic switch 3 (to control the impression actuator 5), automatic switch 4 (to control the compression actuator 5), pressure meter 7, and registration device 6 (registering the signal from pressure meter 7 to measure compression force of the compression actuator). Compression forceps 8 are connected to compression arms 11. Registration device 9 registers a signal from optic meter 10, compression arms 11 and compression feet 14. Computer 15 receives signals from the registration devices and transmits signals, via connector 16, to distance actuators 17.

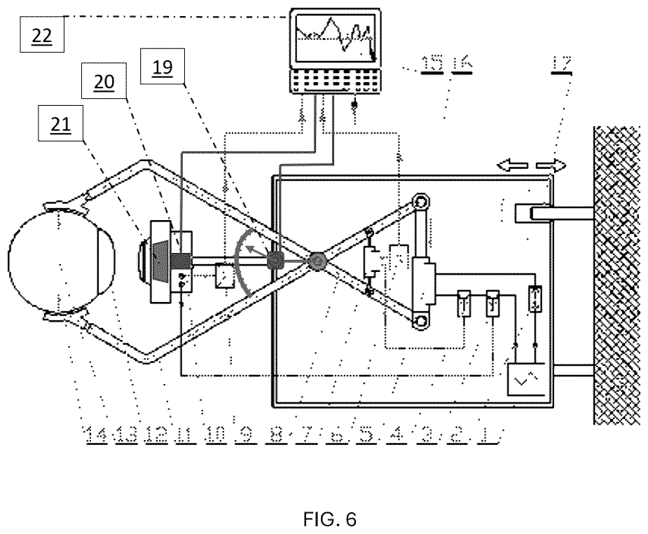

FIG. 6 illustrates a diagram of a tonometer in accordance with disclosed implementations, which includes compression arm-spread angle gauge 19 connected to computer 22, independent axial drive 20 of an optical gauge in the head connected to computer 22, front lens surface reflex camera 21, and calculation algorithm module 22 (e.g., an algorithm programmed into computer 22) to calculate and/or extrapolate physical parameters of the sclera (thickness, axial and bending stiffness). Construction and operation of this tonometer are described in greater detail below.

The additional compression arms-spread sensor will measure the dilation angle when compression feet 14 touch the eyeball wall backwards from the corneal limbus at an equal distance from it. With a larger eyeball and cornea, the sensor will register a larger angle of arm spread to assess the dimensions of the eyeball in the anterior hemisphere based on basic geometrical relationships. Beneficially it is also possible to use the sensor as a linear spacing value controller after the computer inputs the corneal diameter measured by the optical meter in the head. The linear value of arm spacing in the frontal plane of the anterior eyeball wall around the cornea will be converted by the algorithm to a preset angular value of arm spread, allowing the compression feet to be precisely applied to the eyeball surface at a preset distance from the corneal limbus.

Compression arm-spread angle gauge 19 can measure:
casad: compression arms-spread angle distance
ipd: impression points distance (main parameter determining the size of the eyeball)
ipd=cd+2×4.5 mm
cd: corneal diameter=ipd−2×cl/ipd (when measured with a light reflex sensor from the corneal limbus)
cl/ipd: corneal limbus-impression point distance (beneficially 2 and 4.5 mm)

The different angular values measured by the compression arm dilation meter when the compression feet reach several distances from the corneal stub will allow extrapolation of the spatial curvature values of the anterior segment of the eye and the anterior ocular hemisphere. This will allow assessment of the size of the eyeball.

The additional independent axial drive of the optical meter in the head connected to the device computer allows the optical head to maintain a constant and safe distance from the cornea as the compression arms are further extended in the sagittal axis and the independent head of these arms approaches the eyeball. When the eyeball has a greater curvature of the anterior hemisphere, this is usually also associated with a greater curvature of the cornea. This will also advantageously allow points in the sclera to be reached twice, beneficially 2 and 4.5 mm back from the corneal limbus. By measuring the axial head movement of the compression arms reaching these points on the eyeball wall behind the cornea, i the curvature of the anterior hemisphere of the eyeball can be determined. Maintaining the optical measurement head at a uniform distance from the corneal apex, despite the different axial movement of the arm head, is important to accurately measure the change in corneal curvature during the measurement.

An additional camera in the optical head, which captures the reflection from the anterior surface of the lens, allows the degree of porosity of the anterior lens capsule to be determined using known image analysis techniques. The higher the porosity, the lower the lens-ocular adhesion and the weaker the pupillary block as the lens ages and thickens, which naturally leads to increased pupillary block in the aging process. The measurement will help calculate the risk of increased pupillary block.

The IOP adjustment algorithm uses measurements of the geometry of the eyeball, which allows extrapolation/determination of thickness and axial and bending stiffness of a sclera. Furthermore, to determine physical characteristics of the sclera, the algorithm uses variable corneal data measured with an optical tonometer meter (for example, a keratometer and pachymeter). The cornea is a transparent and accessible part of the sclera that can be accurately measured and examined. The principle of correlation of the physical parameters of the cornea and the sclera discovered by the inventors is based on the assumption that the growing eyeball grows proportionally in its entirety, that is, in the anterior and posterior sections. This is proven by measurements of central corneal thickness (CCT), showing that in a larger eyeball (e.g., in myopia) there is also a decrease in corneal thickness stretched in proportion to eyeball enlargement.

The algorithm may also use a value of double IOP measurement on the sclera as a wall of a posterior chamber and a vitreous cavity (beneficially 2 and 4.5 mm backwards from the corneal limbus) to provide data on the stiffness of the ocular wall beyond the cornea. This measurement relative to the value of symmetrical deformation of the cornea (astigmatism) also provides feedback on corneal stiffness. This will allow refinement of the measurement of IOP in the anterior chamber, which can be performed simultaneously using an additional device attached to the measurement head of the patented tonometer (e.g., a non-contact air-puff tonometer or a rebound tonometer). In such a tonometer, appropriate pressure jets compress the eyeball outside the cornea with a jet of air of a preset force to produce variable eyeball deformation, or in another measurement variant, variable and measured air blast pressure to produce the presumed eyewall deformation, which is of course dependent on the prevailing IOP.

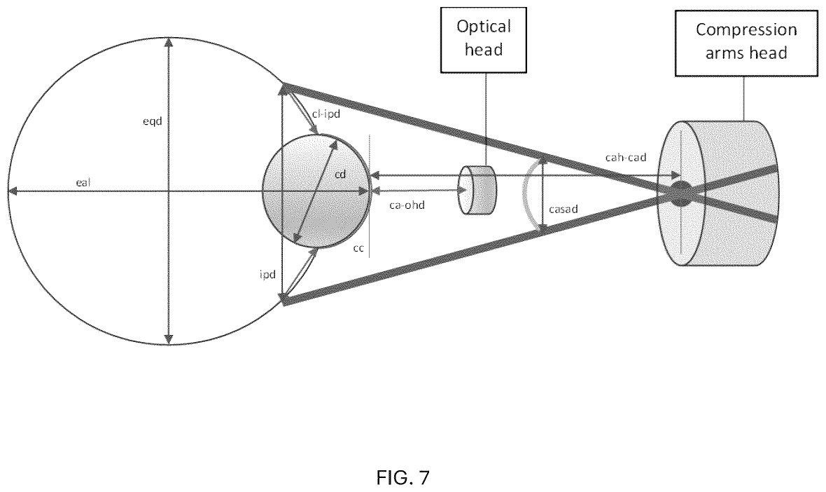

Once the corneal size data measured by the optical head has been entered into the algorithm, the algorithm can determine the angular size of arms spread and scleral compression at a backward distance from the cornea given a percentage to the estimated eyeball size. FIG. 7 is a diagram illustrating measurement of the geometry of the eyeball in accordance with disclosed implementations where:
cc: corneal curvature; keratometrie (brown)
cd: corneal diameter
cl-ipd: corneal limbus-impression point distance (beneficially 4.5 and 2 mm)
ipd: impression points distance
ca-ohd: cornael apex-optical head distance
cah-cad: compression arms head-corneal apex distance
casad: compression arms-spread angle distance
eqd: equator diameter
eal: eyball axial length
a-ca: angle of spread of tonometer compression arms

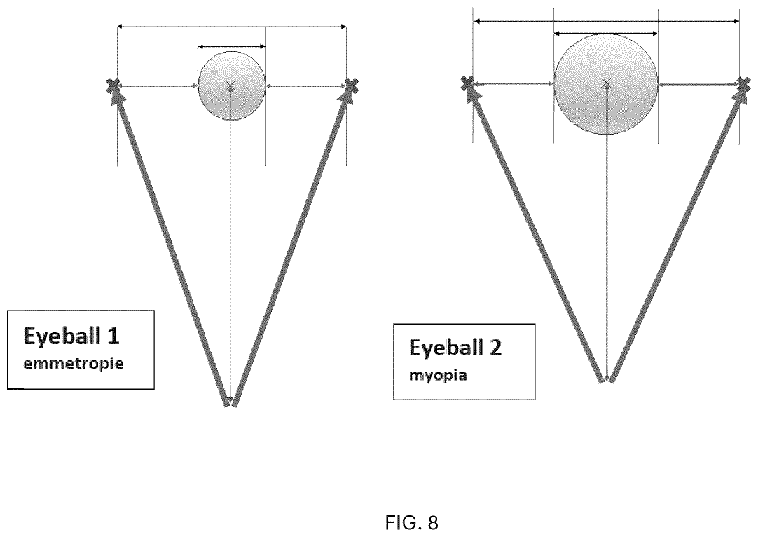

FIG. 8 illustrates the various angular and linear quantities measured by the tonometer in accordance with disclosed implementations, using the compression arm angle gauge and the measurement head's distance sensor from the cornea; data for use in a computational algorithm in estimating the size of the eyeball and the stretch and stenosis of the hydraulic vessels in its walls.
Eyeball 1: little eyeball with a little cornea—(emmetropie)
Eyeball 2: big eyeball with a bigger cornea—(myopia)
cl-ipd 1=cl-ipd 2: cl-ipd: corneal limbus-impression point distance equal
cd 1<cd 2: cd: corneal diameter difference
α-ca1<α-ca2: casad: compression arms-spread angle difference
cah-cad 1>cah-cad 2 cah-cad: compression arms head-corneal apex distance difference
ipd 1<ipd 2: ipd: impression points distance difference

The disclosed implementation include a system and method for measuring intraocular pressure (IOP) that offers a more comprehensive and personalized approach to IOP assessment. The system includes a tonometer head, an optical gauge, a compression arm-spread angle gauge, and a processor. The tonometer head is configured to measure IOP in the posterior chamber and the vitreous cavity of an eye, providing valuable information about pressure differentials within the eye that may not be captured by conventional tonometry methods. The optical gauge is configured to measure corneal data of the eye, which can provide insights into individual anatomical and biomechanical factors that may influence the clinical significance of a given IOP level. The compression arm-spread angle gauge is configured to measure a dilation angle of compression arms, which can provide additional data about the geometry of the eye. The processor is configured to determine geometric parameters of the eye based on the measured corneal data and dilation angle, calculate physical parameters of a sclera of the eye based on the determined geometric parameters, and adjust the measured IOP based on the calculated physical parameters of the sclera. This approach allows for a more nuanced understanding of IOP and its potential impact on ocular health, potentially improving the diagnosis and management of conditions such as glaucoma.

In some aspects, the system for measuring intraocular pressure (IOP) may also include a mechanism for measuring IOP in the anterior chamber of the eye. This can provide a more comprehensive picture of the pressure dynamics within the eye, as it allows for the assessment of pressure in both the anterior and posterior segments of the eye. The anterior chamber IOP measurement can be performed using a variety of techniques. In some cases, a non-contact air-puff tonometer may be used. This type of tonometer measures IOP by directing a brief puff of air onto the surface of the eye and measuring the amount of indentation or flattening of the cornea caused by the air puff. The degree of corneal flattening can be correlated with the IOP.

In other cases, a rebound tonometer may be used to measure IOP in the anterior chamber. A rebound tonometer operates by briefly contacting the surface of the eye with a small probe. The speed at which the probe rebounds from the eye is measured and used to calculate the IOP. Both the non-contact air-puff tonometer and the rebound tonometer offer non-invasive methods for measuring IOP in the anterior chamber, which can complement the measurements taken in the posterior chamber and vitreous cavity to provide a more complete understanding of the pressure dynamics within the eye.

In some aspects, the tonometer head may be configured to measure IOP at two different points on the sclera, beneficially 2 mm and 4.5 mm backwards from the corneal limbus. This dual-point measurement approach can provide valuable information about the pressure distribution within the posterior chamber and vitreous cavity of the eye. The tonometer head may include a pair of compression arms that are configured to contact the sclera at the specified points. The compression arms may be adjustable to accommodate eyes of different sizes and shapes, and to ensure accurate and consistent contact with the sclera at the desired measurement points.

In some cases, the tonometer head may be equipped with sensors that detect the force exerted by the compression arms on the sclera. These sensors can provide real-time feedback about the amount of pressure being applied to the eye, allowing for precise control of the compression force during the IOP measurement process. The data from these sensors can be used by the processor to calculate the IOP in the posterior chamber and vitreous cavity based on the detected compression force and the known geometry of the eye.

In other aspects, the tonometer head may be designed to measure IOP in the posterior chamber and vitreous cavity simultaneously or sequentially. In a simultaneous measurement approach, the tonometer head may include separate sensors for the posterior chamber and vitreous cavity, allowing for the simultaneous detection of IOP in both regions. In a sequential measurement approach, the tonometer head may be moved axially to measure IOP first in the posterior chamber and then in the vitreous cavity, or vice versa. The choice between simultaneous and sequential measurement approaches may depend on various factors, such as the specific clinical needs of the patient, the design of the tonometer, and the desired level of measurement precision.

In some aspects, the system may include an optical gauge configured to measure corneal data of the eye. The corneal data may include, but is not limited to, parameters such as corneal curvature, corneal thickness, and corneal diameter. These parameters can provide valuable insights into the individual anatomical and biomechanical characteristics of the eye, which can influence the clinical significance of a given IOP level.

In some cases, the optical gauge may include a keratometer configured to measure corneal curvature. The keratometer, also known as an ophthalmometer, may operate by projecting a pattern of light onto the cornea and measuring the reflection. The shape and size of the reflected pattern can provide information about the curvature of the cornea, which can be used to calculate the corneal refractive power. This information can be particularly useful in the diagnosis and management of conditions such as astigmatism and keratoconus, which are characterized by abnormal corneal curvature.

In other aspects, the optical gauge may include a pachymeter configured to measure corneal thickness. The pachymeter may operate using ultrasound or optical coherence tomography (OCT) technology. In an ultrasound-based pachymeter, a probe emits high-frequency sound waves that are reflected back from the various layers of the cornea. The time it takes for the sound waves to return to the probe can be used to calculate the thickness of the cornea. In an OCT-based pachymeter, a beam of light is directed onto the cornea and the reflection is analyzed to determine the thickness of the cornea. Corneal thickness can be an important factor in the assessment of IOP, as variations in corneal thickness can influence the accuracy of IOP measurements.

In some embodiments, the optical gauge may be configured to measure both corneal curvature and corneal thickness, providing a comprehensive assessment of the corneal parameters that can influence IOP. The data obtained from the optical gauge can be used by the processor to determine geometric parameters of the eye, calculate physical parameters of the sclera, and adjust the measured IOP accordingly. This integrated approach can provide a more nuanced understanding of IOP and its potential impact on ocular health.

In some aspects, the system may include a compression arm-spread angle gauge configured to measure a dilation angle of compression arms. The dilation angle may be defined as the angle formed by the compression arms when they are spread apart to contact the eye. This angle can provide valuable information about the geometry of the eye, as it can be correlated with the size and shape of the eye. The compression arm-spread angle gauge may be designed to measure the dilation angle accurately and reliably, providing consistent and repeatable measurements.

In some cases, the compression arm-spread angle gauge may be configured to measure the dilation angle when compression feet of the compression arms contact the eye at a predetermined distance from a corneal limbus. The corneal limbus is the border between the cornea and the sclera, and it can serve as a reference point for positioning the compression feet. The predetermined distance may be selected to ensure that the compression feet contact the sclera in a region that is suitable for IOP measurement. In some embodiments, the predetermined distance may be between 3-5 mm posterior to the corneal limbus. This distance may be selected based on anatomical considerations, such as the typical size and shape of the eye, and it may be adjustable to accommodate individual variations in eye geometry.

In other aspects, the compression arm-spread angle gauge may be connected to the processor, allowing the measured dilation angle to be used in the calculation of the geometric parameters of the eye. The processor may use the measured dilation angle, along with the corneal data obtained from the optical gauge, to determine the geometric parameters of the eye. These parameters may include, for example, the size and shape of the eye, the curvature of the cornea, and the thickness of the sclera. The calculated geometric parameters can then be used to adjust the measured IOP, providing a more personalized and accurate assessment of IOP.

In some aspects, the system may include a processor configured to determine geometric parameters of the eye, calculate physical parameters of a sclera, and adjust the measured IOP. The processor may be a dedicated hardware component, or it may be implemented as a software module running on a general-purpose computing device. The processor may be configured to receive data from the tonometer head, the optical gauge, and the compression arm-spread angle gauge, and to process this data to determine the geometric parameters of the eye, calculate the physical parameters of the sclera, and adjust the measured IOP.

In some cases, the processor may determine geometric parameters of the eye based on the measured corneal data and dilation angle. The corneal data may include parameters such as corneal curvature, corneal thickness, and corneal diameter, which can provide insights into the individual anatomical and biomechanical characteristics of the eye. The dilation angle, measured by the compression arm-spread angle gauge, can provide additional data about the geometry of the eye. The processor may use these data to determine geometric parameters of the eye, such as the size and shape of the eye, the curvature of the cornea, and the thickness of the sclera.

In some aspects, the processor may calculate physical parameters of a sclera of the eye based on the determined geometric parameters. The physical parameters of the sclera may include parameters such as thickness, axial stiffness, and bending stiffness. These parameters can provide valuable insights into the biomechanical properties of the eye, which can influence the clinical significance of a given IOP level. The processor may use algorithms, mathematical models, or other computational methods to calculate these physical parameters based on the determined geometric parameters.

In other cases, the processor may adjust the measured IOP based on the calculated physical parameters of the sclera. The adjustment may be performed to account for individual variations in the physical properties of the sclera, which can influence the accuracy of IOP measurements. The processor may use algorithms, mathematical models, or other computational methods to calculate the adjustment factor and apply it to the measured IOP. This adjusted IOP can provide a more personalized and accurate assessment of IOP, potentially improving the diagnosis and management of conditions such as glaucoma.

In some aspects, the system may include an axial drive configured to maintain the optical gauge at a constant distance from a cornea of the eye during measurement. The axial drive may be a mechanical or electronic component that controls the position of the optical gauge relative to the cornea. This can ensure that the optical gauge remains at a consistent distance from the cornea, regardless of the size or shape of the eye or the position of the compression arms. Maintaining a constant distance between the optical gauge and the cornea can help to ensure accurate and consistent measurements of corneal data, which can be critical for determining the geometric parameters of the eye and calculating the physical parameters of the sclera.

In some cases, the axial drive may be configured to adjust the position of the optical gauge in response to the movement of the compression arms. For example, as the compression arms are extended in the sagittal axis to contact the sclera at different points, the axial drive may move the optical gauge axially to maintain a constant distance from the cornea. This can allow the optical gauge to remain in a consistent position relative to the cornea, even as the compression arms are moved to measure IOP at different points on the sclera.

In other aspects, the system may be configured to measure the axial head movement of the compression arms to determine the curvature of the anterior hemisphere of the eyeball. The axial head movement may be defined as the movement of the head of the compression arms in the axial direction, which can provide information about the curvature of the anterior hemisphere of the eyeball. By measuring the axial head movement as the compression arms are extended to contact the sclera at different points, the system can obtain data about the curvature of the anterior hemisphere of the eyeball. This data can be used by the processor to calculate the physical parameters of the sclera, such as its thickness, axial stiffness, and bending stiffness. This can provide valuable information for adjusting the measured IOP, as the physical properties of the sclera can influence the clinical significance of a given IOP level.

In some aspects, the axial drive may employ various mechanical arrangements to maintain the optical gauge at a constant distance from the cornea. One such arrangement may include a threaded rotating shaft. In this configuration, the optical gauge may be mounted on a carriage that moves along the threaded shaft. As the shaft rotates, the carriage moves linearly, allowing for precise control of the optical gauge's position relative to the cornea.

The system may also utilize a rack and pinion mechanism for the axial drive. In this arrangement, a toothed rack may be attached to the optical gauge, while a motorized pinion gear engages with the rack. As the pinion rotates, it drives the rack linearly, moving the optical gauge to maintain the desired distance from the cornea.

In some cases, the axial drive may incorporate a linear actuator. This may include an electric motor that drives a lead screw or ball screw, converting rotational motion into linear motion. The optical gauge may be attached to the moving part of the actuator, allowing for smooth and precise positioning.

Another potential arrangement for the axial drive may involve a pneumatic or hydraulic cylinder. In this configuration, compressed air or fluid may be used to extend or retract a piston, which in turn moves the optical gauge. This approach may offer advantages in terms of speed and force control.

The system may also employ a belt-driven mechanism for the axial drive. A toothed belt may be connected to the optical gauge, with the belt looped around pulleys driven by a motor. As the motor rotates, it moves the belt, which in turn adjusts the position of the optical gauge.

In some embodiments, the axial drive may utilize a piezoelectric actuator. This type of actuator relies on the piezoelectric effect to produce very small, precise movements. Multiple piezoelectric elements may be stacked to achieve larger ranges of motion while maintaining high precision.

The axial drive may also incorporate a voice coil actuator, similar to those used in loudspeakers. This type of actuator may provide rapid, precise movements with low friction, which can be beneficial for maintaining a constant distance between the optical gauge and the cornea.

In some cases, the axial drive may use a combination of these mechanical arrangements to achieve the desired performance characteristics. For example, a coarse positioning mechanism like a threaded shaft may be combined with a fine positioning mechanism like a piezoelectric actuator to provide both a large range of motion and high precision.

In some aspects, the system may include a camera configured to capture an image of an anterior surface of a lens of the eye. This camera may be integrated into the tonometer head or it may be a separate component that is positioned to capture an image of the anterior lens surface. The camera may be any type of camera suitable for capturing high-resolution images of the eye, such as a digital camera or a charge-coupled device (CCD) camera. The camera may be equipped with a lens system that allows for the focusing of the image on the anterior lens surface, and it may also include features such as image stabilization and automatic focus adjustment to ensure clear and sharp images.

In some cases, the camera may be configured to capture an image of the anterior lens surface under specific lighting conditions. For example, the camera may be used in conjunction with a light source that illuminates the anterior lens surface in a particular way to enhance the visibility of certain features. The light source may be a part of the tonometer head, or it may be a separate component. The light source may emit light of a specific wavelength or a range of wavelengths that are suitable for imaging the anterior lens surface.

In some aspects, the system may be configured to determine a degree of porosity of an anterior lens capsule based on the image captured by the camera. The anterior lens capsule is a thin, transparent structure that encloses the lens of the eye, and its porosity can influence the degree of pupillary block, which in turn can affect IOP. The processor may analyze the captured image to identify features indicative of the porosity of the anterior lens capsule, such as the presence of pores or other irregularities on the surface of the capsule. The processor may use image processing techniques, such as edge detection, texture analysis, or pattern recognition, to analyze the image and determine the degree of porosity of the anterior lens capsule.

In some cases, the processor may use the determined degree of porosity of the anterior lens capsule to adjust the measured IOP. For example, if the anterior lens capsule is determined to have a high degree of porosity, this may indicate a lower risk of increased pupillary block, and the processor may adjust the measured IOP downwards accordingly. Conversely, if the anterior lens capsule is determined to have a low degree of porosity, this may indicate a higher risk of increased pupillary block, and the processor may adjust the measured IOP upwards accordingly. This adjustment can provide a more personalized and accurate assessment of IOP, potentially improving the diagnosis and management of conditions such as glaucoma.

In some aspects, the method of measuring IOP may involve a series of steps that include data collection, parameter calculation, and IOP adjustment. The data collection step may involve measuring IOP in the posterior chamber and the vitreous cavity of an eye using a tonometer. This measurement can provide valuable information about the pressure dynamics within the eye, which may not be captured by conventional tonometry methods. The data collection step may also involve measuring corneal data of the eye using an optical gauge. The corneal data may include parameters such as corneal curvature, corneal thickness, and corneal diameter, which can provide insights into the individual anatomical and biomechanical characteristics of the eye. Additionally, the data collection step may involve measuring a dilation angle of compression arms using a compression arm-spread angle gauge. The dilation angle can provide additional data about the geometry of the eye, as it can be correlated with the size and shape of the eye.

In some cases, the parameter calculation step may involve determining geometric parameters of the eye based on the measured corneal data and dilation angle. The geometric parameters may include parameters such as the size and shape of the eye, the curvature of the cornea, and the thickness of the sclera. These parameters can provide valuable insights into the biomechanical properties of the eye, which can influence the clinical significance of a given IOP level. The parameter calculation step may also involve calculating physical parameters of a sclera of the eye based on the determined geometric parameters. The physical parameters may include parameters such as thickness, axial stiffness, and bending stiffness. These parameters can provide valuable insights into the biomechanical properties of the eye, which can influence the clinical significance of a given IOP level.

In some aspects, the IOP adjustment step may involve adjusting the measured IOP based on the calculated physical parameters of the sclera. The adjustment may be performed to account for individual variations in the physical properties of the sclera, which can influence the accuracy of IOP measurements. The IOP adjustment step may involve applying a correction factor to the measured IOP based on the calculated physical parameters of the sclera. This correction factor can be calculated using algorithms, mathematical models, or other computational methods. The adjusted IOP can provide a more personalized and accurate assessment of IOP, potentially improving the diagnosis and management of conditions such as glaucoma.

In some cases, the method may involve repeating the data collection, parameter calculation, and IOP adjustment steps multiple times to obtain a series of IOP measurements. These measurements can be used to monitor changes in IOP over time, which can provide valuable information for the management of conditions such as glaucoma. The method may also involve comparing the adjusted IOP measurements with a reference range or threshold to determine whether the IOP is within a normal range or whether it indicates a risk of ocular disease.

In some aspects, the processor may determine geometric parameters of the eye based on the measured corneal data and dilation angle. The corneal data may include parameters such as corneal curvature, corneal thickness, and corneal diameter, which can provide insights into the individual anatomical and biomechanical characteristics of the eye. The dilation angle, measured by the compression arm-spread angle gauge, can provide additional data about the geometry of the eye. The processor may use these data to determine geometric parameters of the eye, such as the size and shape of the eye, the curvature of the cornea, and the thickness of the sclera.

In some cases, the processor may extrapolate the spatial curvature values of the anterior segment of the eye and the anterior ocular hemisphere based on the measured corneal data and dilation angle. This extrapolation may involve applying mathematical models or algorithms that take into account the measured corneal data and dilation angle, as well as other known anatomical and biomechanical characteristics of the eye. The extrapolated spatial curvature values can provide valuable information about the geometry of the eye, which can be used to calculate the physical parameters of the sclera.

In some aspects, the compression arm-spread angle gauge may be used as a linear spacing value controller. This may involve using the measured dilation angle to control the spacing between the compression feet of the compression arms. The processor may input the measured corneal diameter into the compression arm-spread angle gauge, which can then adjust the spacing of the compression feet to achieve a preset angular value of arm spread. This can ensure that the compression feet are precisely applied to the sclera at a preset distance from the corneal limbus, which can improve the accuracy of the IOP measurement.

In some cases, the processor may use the measured dilation angle and the input corneal diameter to calculate the linear value of arm spacing in the frontal plane of the anterior eyeball wall around the cornea. This calculated linear value of arm spacing can be converted by the processor into a preset angular value of arm spread, which can be used to adjust the position of the compression feet on the sclera. This can allow for precise control of the compression force applied to the sclera, which can improve the accuracy of the IOP measurement.

In other aspects, the processor may use the measured dilation angle and the input corneal diameter to extrapolate the geometry of the entire eyeball, the degree of wall thinning with subsequent constriction of the hydraulic vessels in the fundus of the eye, and the limiting pressure for a given eyeball. This extrapolation may involve applying mathematical models or algorithms that take into account the measured dilation angle, the input corneal diameter, and other known anatomical and biomechanical characteristics of the eye. The extrapolated geometric parameters can provide valuable information about the geometry of the eye, which can be used to calculate the physical parameters of the sclera.

In some aspects, the processor may calculate physical parameters of the sclera based on the determined geometric parameters. These physical parameters may include, but are not limited to, the thickness, axial stiffness, and bending stiffness of the sclera. The thickness of the sclera may be estimated based on the measured corneal data and dilation angle, as well as known anatomical and biomechanical characteristics of the eye. The axial stiffness and bending stiffness of the sclera may be calculated based on the estimated thickness and other geometric parameters of the eye, such as the size and shape of the eye and the curvature of the cornea.

In some cases, the processor may use the principle of correlation between the physical parameters of the cornea and the sclera to estimate the physical parameters of the sclera. This principle is based on the assumption that the eye grows proportionally in its entirety, meaning that changes in the size and shape of the cornea are likely to be accompanied by corresponding changes in the size and shape of the sclera. For example, if the cornea is determined to be thicker or more curved, this may suggest that the sclera is also likely to be thicker or more curved. Similarly, if the cornea is determined to be thinner or less curved, this may suggest that the sclera is also likely to be thinner or less curved. By applying this principle of correlation, the processor can estimate the physical parameters of the sclera based on the measured corneal data and dilation angle.

In other aspects, the processor may use mathematical models or algorithms to calculate the physical parameters of the sclera. These models or algorithms may take into account the determined geometric parameters of the eye, the measured corneal data and dilation angle, and the principle of correlation between the physical parameters of the cornea and the sclera. The models or algorithms may also incorporate known anatomical and biomechanical characteristics of the eye, such as the typical size and shape of the eye, the typical curvature of the cornea, and the typical thickness of the sclera. By applying these models or algorithms, the processor can calculate the physical parameters of the sclera with a high degree of accuracy and precision.

In some cases, the processor may adjust the measured IOP based on the calculated physical parameters of the sclera. This adjustment may be performed to account for individual variations in the physical properties of the sclera, which can influence the accuracy of IOP measurements. For example, if the sclera is determined to be thicker or stiffer, this may suggest that the eye is more resistant to changes in IOP, and the processor may adjust the measured IOP downwards accordingly. Conversely, if the sclera is determined to be thinner or less stiff, this may suggest that the eye is less resistant to changes in IOP, and the processor may adjust the measured IOP upwards accordingly. This adjustment can provide a more personalized and accurate assessment of IOP, potentially improving the diagnosis and management of conditions such as glaucoma.

In some aspects, the processor may utilize an algorithm to adjust the measured IOP based on the calculated physical parameters of the sclera. The algorithm may take into account parameters such as the thickness, axial stiffness, and bending stiffness of the sclera. These parameters can provide valuable insights into the biomechanical properties of the eye, which can influence the clinical significance of a given IOP level. The algorithm may apply a correction factor to the measured IOP based on these calculated physical parameters of the sclera. This correction factor can be calculated using mathematical models or other computational methods. The adjusted IOP can provide a more personalized and accurate assessment of IOP, potentially improving the diagnosis and management of conditions such as glaucoma.

In some cases, the algorithm may also use corneal astigmatism data to provide feedback on corneal stiffness. Corneal astigmatism refers to a condition in which the cornea has an irregular shape, often resulting in blurred or distorted vision. The degree of corneal astigmatism can be measured using the optical gauge, and this data can be used by the algorithm to estimate the stiffness of the cornea. The stiffness of the cornea can influence the IOP measurement, as a stiffer cornea may resist deformation under pressure more than a less stiff cornea. By taking into account the corneal astigmatism data, the algorithm can provide a more accurate adjustment of the measured IOP.

In other aspects, the algorithm may be configured to adjust the measured IOP based on the calculated physical parameters of the sclera and the corneal astigmatism data in a variety of ways. For example, the algorithm may apply different correction factors to the measured IOP depending on the calculated thickness, axial stiffness, and bending stiffness of the sclera, as well as the degree of corneal astigmatism. The algorithm may also take into account other factors, such as the size and shape of the eye, the curvature of the cornea, and the dilation angle of the compression arms, when adjusting the measured IOP. This can allow for a more nuanced and personalized adjustment of the measured IOP, potentially improving the accuracy and clinical relevance of the IOP measurement.

The instructions may also include routines for determining geometric parameters of the eye based on the received corneal data and dilation angle, calculating physical parameters of a sclera of the eye based on the determined geometric parameters, and adjusting the received IOP measurements based on the calculated physical parameters of the sclera. These routines may involve applying mathematical models or algorithms to the received data to calculate the geometric and physical parameters, and to adjust the IOP measurements.

In some aspects, the instructions may include routines for capturing an image of an anterior surface of a lens of the eye and determining a degree of porosity of an anterior lens capsule based on the captured image. These routines may involve interfacing with a camera to capture the image, and applying image processing techniques to analyze the image and determine the degree of porosity.

In some cases, the instructions may include routines for maintaining an optical gauge at a constant distance from a cornea of the eye during measurement. These routines may involve interfacing with an axial drive to control the position of the optical gauge, and applying control algorithms to maintain the desired distance.

In other aspects, the instructions may include routines for repeating the data collection, parameter calculation, and IOP adjustment steps multiple times to obtain a series of IOP measurements. These routines may involve looping or iterative structures in the programming code, and they may include provisions for storing the series of IOP measurements for later analysis or comparison.

In some cases, the instructions may include routines for comparing the adjusted IOP measurements with a reference range or threshold to determine whether the IOP is within a normal range or whether it indicates a risk of ocular disease. These routines may involve applying comparison operators or decision-making structures in the programming code, and they may include provisions for generating alerts or notifications if the IOP is outside the normal range or exceeds a certain threshold.

In some aspects, the system may include a camera configured to capture an image of an anterior surface of a lens of the eye. This camera may be integrated into the tonometer head or it may be a separate component that is positioned to capture an image of the anterior lens surface. The camera may be any type of camera suitable for capturing high-resolution images of the eye, such as a digital camera or a charge-coupled device (CCD) camera. The camera may be equipped with a lens system that allows for the focusing of the image on the anterior lens surface, and it may also include features such as image stabilization and automatic focus adjustment to ensure clear and sharp images.

In some cases, the camera may be configured to capture an image of the anterior lens surface under specific lighting conditions. For example, the camera may be used in conjunction with a light source that illuminates the anterior lens surface in a particular way to enhance the visibility of certain features. The light source may be a part of the tonometer head or it may be a separate component. The light source may emit light of a specific wavelength or a range of wavelengths that are suitable for imaging the anterior lens surface.

In some aspects, the system may be configured to determine a degree of porosity of an anterior lens capsule based on the image captured by the camera. The anterior lens capsule is a thin, transparent structure that encloses the lens of the eye, and its porosity can influence the degree of pupillary block, which in turn can affect IOP. The processor may analyze the captured image to identify features indicative of the porosity of the anterior lens capsule, such as the presence of pores or other irregularities on the surface of the capsule. The processor may use image processing techniques, such as edge detection, texture analysis, or pattern recognition, to analyze the image and determine the degree of porosity of the anterior lens capsule.

In some cases, the processor may use the determined degree of porosity of the anterior lens capsule to adjust the measured IOP. For example, if the anterior lens capsule is determined to have a high degree of porosity, this may indicate a lower risk of increased pupillary block, and the processor may adjust the measured IOP downwards accordingly. Conversely, if the anterior lens capsule is determined to have a low degree of porosity, this may indicate a higher risk of increased pupillary block, and the processor may adjust the measured IOP upwards accordingly. This adjustment can provide a more personalized and accurate assessment of IOP, potentially improving the diagnosis and management of conditions such as glaucoma.

In some aspects, the system may include additional or alternative components to those described above. For instance, the tonometer head may include multiple sensors for measuring IOP at different points within the eye. These sensors may be arranged in various configurations to measure IOP in different regions of the posterior chamber and vitreous cavity. This can provide a more detailed picture of the pressure distribution within the eye, potentially improving the accuracy and clinical relevance of the IOP measurement.

In some cases, the optical gauge may include additional or alternative components for measuring corneal data. For example, the optical gauge may include a corneal topographer for measuring the shape of the cornea, or a specular microscope for examining the corneal endothelium. These additional measurements can provide further insights into the individual anatomical and biomechanical characteristics of the eye, which can influence the clinical significance of a given IOP level.

In other aspects, the compression arm-spread angle gauge may be configured to measure the dilation angle at multiple points along the compression arms. This can provide additional data about the geometry of the eye, as it can reveal variations in the curvature of the eye's surface. This additional data can be used by the processor to refine the calculation of the geometric parameters of the eye and the physical parameters of the sclera.

In some cases, the processor may use different algorithms or mathematical models to calculate the physical parameters of the sclera and adjust the measured IOP. For example, the processor may use machine learning algorithms to model the relationship between the geometric parameters of the eye and the physical parameters of the sclera. These algorithms can be trained on a large dataset of eye measurements, allowing them to accurately predict the physical parameters of the sclera based on the measured geometric parameters.

In other aspects, the system may include additional or alternative mechanisms for maintaining the optical gauge at a constant distance from the cornea during measurement. For example, the system may include a feedback control system that continuously adjusts the position of the optical gauge based on real-time measurements of the distance between the optical gauge and the cornea. This can ensure that the optical gauge remains at a consistent distance from the cornea, regardless of variations in the size or shape of the eye or the position of the compression arms.

In some cases, the system may include additional or alternative mechanisms for capturing an image of the anterior surface of the lens of the eye. For example, the system may include a slit lamp biomicroscope or an optical coherence tomography (OCT) device for imaging the anterior surface of the lens. These devices can provide high-resolution images of the lens surface, which can be used to determine the degree of porosity of the anterior lens capsule.

In other aspects, the system may include additional or alternative mechanisms for determining the degree of porosity of the anterior lens capsule. For example, the system may include a software module that uses image processing techniques to analyze the captured image and identify features indicative of the porosity of the anterior lens capsule. This software module may use techniques such as edge detection, texture analysis, or pattern recognition to analyze the image and determine the degree of porosity of the anterior lens capsule.

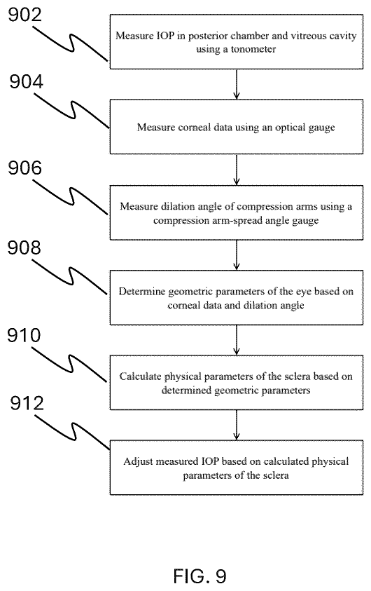

FIG. 9 illustrates a flowchart for a method of measuring and adjusting intraocular pressure (IOP). The process begins at 902 with measuring IOP in the posterior chamber and vitreous cavity using a tonometer. Following this, corneal data is measured using an optical gauge at 904. The next step involves measuring the dilation angle of compression arms using a compression arm-spread angle gauge at 906. The process then advances to determining geometric parameters of the eye based on the previously gathered corneal data and dilation angle at 908. This determination serves as input for step 910 which calculates the physical parameters of the sclera based on the determined geometric parameters. At 912, the initially measured IOP is adjusted based on the calculated physical parameters of the sclera. This final step integrates the data collected and calculations performed in the preceding steps to refine the IOP measurement.

The sequence of steps can be linear, with each step dependent on the data or results obtained from the preceding step. The method incorporates specific gauges and calculation techniques to refine the accuracy of IOP measurements. The use of specialized gauges and the methodical calculation of eye parameters are features of this process, contributing to the precision of the IOP adjustments.

In some aspects, the system may include a non-transitory computer-readable medium storing instructions that, when executed by a processor, cause the processor to perform a method of measuring intraocular pressure (IOP). The computer-readable medium may be any type of medium that can store data in a form that can be read by a computer. Examples of such media include, but are not limited to, hard drives, solid-state drives, flash memory, optical discs, magnetic tapes, and server memory.

The instructions stored on the computer-readable medium may include a series of commands or routines that are executed by the processor to perform the method of measuring IOP. These instructions may be written in any suitable programming language, such as C++, Java, Python, or assembly language, and they may be compiled or interpreted as needed to execute on the processor.

In some cases, the instructions may include routines for receiving IOP measurements from a posterior chamber and a vitreous cavity of an eye, receiving corneal data measurements of the eye, and receiving a dilation angle measurement of compression arms. These routines may involve interfacing with the tonometer head, the optical gauge, and the compression arm-spread angle gauge to obtain the necessary data.

In some aspects, the system may include a non-transitory computer-readable medium storing instructions that, when executed by a processor, cause the processor to perform a method of measuring intraocular pressure (IOP). The computer-readable medium may be any type of medium that can store data in a form that can be read by a computer. Examples of such media include, but are not limited to, hard drives, solid-state drives, flash memory, optical discs, magnetic tapes, and server memory.

The instructions stored on the computer-readable medium may include a series of commands or routines that are executed by the processor to perform the method of measuring IOP. These instructions may be written in any suitable programming language, such as C++, Java, Python, or assembly language, and they may be compiled or interpreted as needed to execute on the processor.

In some cases, the instructions may include routines for receiving IOP measurements from a posterior chamber and a vitreous cavity of an eye, receiving corneal data measurements of the eye, and receiving a dilation angle measurement of compression arms. These routines may involve interfacing with the tonometer head, the optical gauge, and the compression arm-spread angle gauge to obtain the necessary data.

In some cases, the system may include additional or alternative mechanisms for adjusting the measured IOP based on the calculated physical parameters of the sclera. For example, the system may include a feedback control system that continuously adjusts the measured IOP based on real-time measurements of the physical parameters of the sclera. This can provide a more personalized and accurate assessment of IOP, potentially improving the diagnosis and management of conditions such as glaucoma.

What is claimed is:

1. A system for measuring intraocular pressure (IOP), comprising:
a tonometer head configured to measure IOP in a posterior chamber and a vitreous cavity of an eye;
an optical gauge configured to measure corneal data of the eye;
a compression arm-spread angle gauge configured to measure a dilation angle of compression arms; and
a processor configured to determine geometric parameters of the eye based on the measured corneal data and dilation angle, calculate physical parameters of a sclera of the eye based on the determined geometric parameters, and adjust the measured IOP based on the calculated physical parameters of the sclera.

2. The system of claim 1, wherein the optical gauge comprises a keratometer configured to measure corneal curvature.

3. The system of claim 1, wherein the optical gauge comprises a pachymeter configured to measure corneal thickness.

4. The system of claim 1, wherein the compression arm-spread angle gauge is configured to measure the dilation angle when compression feet of the compression arms contact the eye at a predetermined distance from a corneal limbus.

5. The system of claim 4, wherein the predetermined distance is between 3-5 mm posterior to the corneal limbus.

6. The system of claim 1, further comprising an axial drive configured to maintain the optical gauge at a constant distance from a cornea of the eye during measurement.

7. The system of claim 1, further comprising a camera configured to capture an image of an anterior surface of a lens of the eye to determine a degree of porosity of an anterior lens capsule.

8. A method of measuring intraocular pressure (IOP), comprising:
measuring IOP in a posterior chamber and a vitreous cavity of an eye using a tonometer;
measuring corneal data of the eye using an optical gauge;
measuring a dilation angle of compression arms using a compression arm-spread angle gauge;
determining geometric parameters of the eye based on the measured corneal data and dilation angle;
calculating physical parameters of a sclera of the eye based on the determined geometric parameters; and
adjusting the measured IOP based on the calculated physical parameters of the sclera.

9. The method of claim 8, wherein measuring the corneal data comprises measuring corneal curvature using a keratometer.

10. The method of claim 9, wherein measuring the corneal data further comprises measuring corneal thickness using a pachymeter.

11. The method of claim 8, wherein measuring the dilation angle comprises measuring the dilation angle when compression feet of the compression arms contact the eye at a predetermined distance from a corneal limbus.

12. The method of claim 11, wherein the predetermined distance is between 3-5 mm posterior to the corneal limbus.

13. The method of claim 8, further comprising maintaining an optical gauge at a constant distance from a cornea of the eye during measurement using an axial drive.

14. The method of claim 13, further comprising capturing an image of an anterior surface of a lens of the eye to determine a degree of porosity of an anterior lens capsule.

15. A non-transitory computer-readable medium storing instructions that, when executed by a processor, cause the processor to perform a method of measuring intraocular pressure (IOP), the method comprising:
receiving IOP measurements from a posterior chamber and a vitreous cavity of an eye;
receiving corneal data measurements of the eye;
receiving a dilation angle measurement of compression arms;
determining geometric parameters of the eye based on the received corneal data and dilation angle;
calculating physical parameters of a sclera of the eye based on the determined geometric parameters; and
adjusting the received IOP measurements based on the calculated physical parameters of the sclera.

16. The non-transitory computer-readable medium of claim 15, wherein the corneal data measurements comprise corneal curvature and corneal thickness.

17. The non-transitory computer-readable medium of claim 15, wherein the method further comprises receiving an image of an anterior surface of a lens of the eye.

18. The non-transitory computer-readable medium of claim 17, wherein the method further comprises determining a degree of porosity of an anterior lens capsule based on the received image.

19. The non-transitory computer-readable medium of claim 15, wherein calculating the physical parameters of the sclera comprises determining a thickness, axial stiffness, and bending stiffness of the sclera.

20. The non-transitory computer-readable medium of claim 19, wherein adjusting the received IOP measurements comprises applying a correction factor based on the determined thickness, axial stiffness, and bending stiffness of the sclera.
